(19) United States
(12) Patent Application Publication
(10) Pub. No.: US 2004/0229771 A1
(43) Pub. Date: Nov. 18, 2004

(54) Method of reducing decline of or preserving residual renal function

(76) Inventors: Reinhold Deppisch, Hechingen (DE); Anders Wieslander, Lund (SE); Marianne Haag-Weber, Landshut (DE); Ulrike Haug, Hechingen (DE)

Correspondence Address: GREENLEE WINNER AND SULLIVAN P C, 5370 MANHATTAN CIRCLE, SUITE 201, BOULDER, CO, 80303, US

(21) Appl. No.: 10/838,870

(22) Filed: May 3, 2004

Related U.S. Application Data
(60) Provisional application No. 60/471,476, filed on May 15, 2003.
(60) Provisional application No. 60/509,836, filed on Oct. 8, 2003.

(51) Int. Cl. A61K 31/00

(52) U.S. Cl. 514/1

(57) ABSTRACT

The present invention relates to a method of reducing decline of renal function or preserving residual renal function of a subject, particularly, those with impaired renal function, by regulating the amount of carbohydrate-derived toxins that is administered thereto, or is present in the body of such a subject. The method involves administering a product containing reduced amount of carbohydrate-derived toxins to such a subject or a product capable of removing or reducing such toxins in the body of the subject upon administration. Examples of the products useful for the invention include various dialysis fluids, particularly peritoneal dialysis fluids, and food or nutritional fluids containing reduced amount of carbohydrate-derived toxins that can be administered to a patient with impaired renal function.

### CROSS REFERENCE TO RELATED APPLICATIONS

This application claims priority from U.S. Provisional Applications No. 60/471,476 and No. 60/509,836 filed May 15, 2003 and Oct. 8, 2003, respectively, which are incorporated herein in their entirety, to the extent not inconsistent with the present application.

### FIELD OF THE INVENTION

The present invention relates to a method of reducing decline of renal function or preserving residual renal function of a patient, in particular, in association with peritoneal dialysis or in clinical situations with deteriorating renal function.

### BACKGROUND OF THE INVENTION

Peritoneal dialysis is an alternative to hemo-dialysis for renal replacement therapy. Peritoneal dialysis is a method for exchanging solutes and fluid through the capillary vessels of a patient's peritoneum. To obtain the transport, a hypertonic solution is infused into and withdrawn from the peritoneal cavity of the patient via a catheter several times a day. The principle for this method is diffusion and convection mainly facilitated by the use of an osmotic gradient. The osmotic gradient is established by the use of hyper-osmotic concentration of different substances, e.g. a carbohydrate, such as glucose or glucose polymers. Peritoneal dialysis has many advantages, e.g., in general, no special apparatus is required, which makes it possible to be a self care treatment. It gives less influence on the hemodynamics or stress to blood components because extracorporeal circulation of the patient's blood is not necessary, and further the peritoneal dialysis is a continuous treatment and therefore more similar to the continuous metabolic function of the kidneys.

Peritoneal dialysis is usually classified as continuous ambulatory peritoneal dialysis (CAPD), intermittent peritoneal dialysis (IPD), and continuous cyclic peritoneal dialysis (CCPD) or automated peritoneal dialysis (APD).

In PD a catheter is permanently implanted in the abdominal wall of the patient and about 1.5 to 2.5 l of the dialysis fluid is introduced via the catheter into the peritoneal cavity and exchanged several times per day. The peritoneal cavity is flooded with this fluid, left for an appropriate dwell period and then drained out in a waste bag. Removal of solutes and water takes place across the peritoneum, which acts as a semi-permeable membrane.

The dialysis fluid commonly used for peritoneal dialysis is an aqueous solution comprising an osmotic agent such as a carbohydrate, e.g. glucose, glucose polymers and the like, electrolytes such as sodium, potassium, calcium, magnesium, and organic acid salts such as sodium lactate, sodium bicarbonate, or sodium pyruvate as buffer source. The components of these peritoneal dialysis fluids are selected to control the levels of electrolytes or the acid-base equilibrium, to remove waste materials and to efficiently carry out ultrafiltration, i.e., fluid removal.

It is known that carbohydrates are degraded during heat sterilisation and storage. Accordingly, conventional PD fluids with glucose as osmotic agent generally contain large amounts of toxic glucose degradation products (GDPs) such as 3-deoxyglucosone (3-DG), acetaldehyde, 3,4-dideoxyglucosone-3-ene (3,4-DGE), and methylglyoxal, which are formed during heat sterilisation and storage.

Glucose degradation products (GDPs) are cytotoxic, induce pro-inflammatory activation signals, and promote formation of advanced glycation end products (AGE) known to accelerate arteriosclerosis, i.e., vascular damage in dialysis patients. To date only a part of all GDPs in PD fluids has been chemically characterised. Recently 3,4-dideoxyglucosone-3-ene (3,4-DGE) was identified as the GDP component with the so far strongest biological activity and cytotoxicity (1).

3,4-DGE is shown to have immunosuppressive effects in cell culture experiments (2). The 3,4-DGE concentrations in conventional PD fluids are in the range of 10-40 μM depending on glucose concentration, whereas the 3,4-DGE concentration in Gambrosol™ trio is only in the range of 0.5-3 μM (3). The total amount of so far identified GDP species (acetaldehyde, formaldehyde, glyoxal, methylglyoxal, 3-deoxyglucosone, 3,4-dideoxyglucosone-3-ene) in conventional fluids is in the range of approximately 450 μM (assuming 4.0 w/v % glucose in the fluid) whereas the Gambrosol™ trio fluids contain <50 μM GDPs.

GDPs are alpha-dicarbonyl compounds, which easily react with proteins to form advanced glycated end products (AGEs). AGEs are non-enzymatically modified proteins according to the Amadori- and Maillard reactions. They are produced under conditions of elevated carbonyl and oxidative stress, e.g. in diabetic or ESRD patients. In ESRD (End Stage Renal Disease=renal disease requiring extracorporeal therapeutic removal of uremic toxins and fluid overload) patients with impaired renal function elimination of components of the AGE-species is impaired, which adds to the accumulation of AGEs in the body. In general, it is believed that the compromised or reduced renal function is associated with reduced metabolic degradation and excretion of AGE species.

Enhanced generation and impaired elimination of AGEs lead to increased protein and lipoprotein deposition, inactivation of nitric oxide, promotion of matrix protein synthesis and glomerular sclerosis. These factors and related clinical complications contribute significantly to the elevated cardiovascular mortality in these patients compared to healthy controls.

There are also local adverse effects of GDP in the peritoneal tissue. Nakayama et al. showed a significant accumulation of AGE in the peritoneum, i.e., at the vascular walls, in ESRD patients after 34 months and further pronounced after 84 months of peritoneal dialysis (4).

Recently it has been shown that GDPs in PD fluids can be resorbed from the peritoneal cavity and act systemically. Comparison of plasma AGE levels in PD patients treated with conventional PD fluids containing high GDP levels with those treated with newly developed low-GDP PD fluids revealed that the patients treated with high-GDP PD fluids had significantly elevated AGE concentrations compared to those treated with low-GDP PD fluids (5). This indicates that it is important to use a peritoneal dialysis fluid with as low GDPs as possible. Such a product is available, for example, Gambrosol™ trio.

Different physicians have further investigated the importance of preserving the residual renal function (RRF) of patients suffering from renal failure. Lysaght et al. studied the decline of residual renal function in ESRD patients in dependence of the primary underlying renal diseases, e.g. diabetic nephropathy versus other non-diabetic reasons (such as IgA-nephropathy or glomerulonephritis) and the mode of therapy of end stage renal disease (=terminal phase renal disease, where the residual function reaches a critically low level with severe life threatening symptoms), i.e. hemodialysis (HD) versus PD treatment. The major findings on the time course of the RRF decay clearly indicate the general problem of start of renal replacement therapy: the decline of residual renal function from the onset of ESRD therapy is greater in HD than in PD. Furthermore, in PD, the residual renal function declines fastest in patient with diabetic nephropathy as underlying renal disease (6).

Bargman et al. published an article in which the authors re-analysed data from the CANUSA study to investigate the assumption that renal and peritoneal clearance are comparable and additive. The results were that the residual renal function has a much greater effect on the mortality risk than peritoneal clearance (7).

Hypertension in ESRD patients is considered as a progressive disease primarily related to falling glomerular filtration rate. Thus, the preservation of residual renal function can improve blood pressure control and possibly modify cardiovascular risk (8).

Shemin et al. analysed data obtained from 990 PD patients. They concluded that even low levels of residual renal function are associated with reduced mortality risk and a better nutritional state (9).

Venkataraman and Nolph gave an overview of recent findings on residual renal function. They emphasize the fact that the maintenance of residual renal function has an effect on mortality, intake of proteins and calories, preservation of non-excretory endocrine functions of the kidney, (e.g. vitamin D and erythropoietin production and calcium and phosphorus homeostasis and EPO production). At the initiation of dialysis the residual renal function is according to USRDS data registry in the range of 6.6-8.0 ml/min. Each 1 ml/min in residual renal function is equivalent to significant blood purification effect, i.e. approximately 0.25 to 0.3 Kt/V or approximately 10 l/week creatinine clearance (10).

Rocco et al. showed that residual renal function is an important predictor of 1-year mortality in chronic peritoneal dialysis patients (11).

The studies described above indicate that there is a long-felt need in the art for a method by which the decline of renal function can be prevented or minimized or the residual renal function can be preserved in a patient, particularly those suffering from deteriorating renal function. The invention disclosed herein provides a method meeting that need. The inventors discovered that the carbohydrate-derived toxins present at significant levels in the peritoneal dialysis (PD) fluids commonly used in the art are factors contributing to the decline of renal function. Accordingly, the invention provides a method of reducing decline of renal function or preserving residual renal function by administering a product that contains less carbohydrate-derived toxins. The advantages of the invention will become apparent in the description below.

### BRIEF DESCRIPTION OF THE DRAWINGS

FIGS. 1A and 1B demonstrate that the patients (“trial group”) who had been treated with PD solution (Gambrosol™ trio) containing a low amount of glucose degradation product (GDP) showed a significantly less decline in the residual renal function (RRF) compared to the standard group which had been treated with a PD-solution containing 1.5 w/v % glucose and >350 μM GDPs. FIG. 1A shows the results of the standard group indicating that the residual renal function declined over time (up to 25 months) significantly. FIG. 1B shows the results of the trial group indicating that the residual renal function remained approximately the same over the duration of the trial period (25 months). Details of the analyses are provided in the text.

### SUMMARY OF THE INVENITON

The present invention provides a method of reducing decline of renal function of a subject or preserving residual renal function of a subject with impaired renal function by regulating the amount of carbohydrate-derived toxins, present in a product that is administered thereto, or present in the body of the subject. The invention is based on the discovery that the currently available peritoneal dialysis fluids having glucose as osmotic agent contain high levels of glucose degradation products (GDPs) and that these GDPs cause rapid decline of renal function when administered to a subject, particularly those with impaired renal function. Accordingly, the invention provides new methods of treating a subject by administering a product with reduced amount of carbohydrate-derived toxins such that the decline of renal function in the subject is minimized or the residual renal function is preserved.

The products useful for the method of the invention include without limitation a variety of dialysis fluids, food or nutritional fluids, particularly those of cystein-rich foodstuff and beverages, containing no, low or reduced amount of carbohydrate-derived toxins, or other agents capable of removing or reducing the carbohydrate-derived toxins present in the body of a subject. The dialysis fluids, food and the nutritional fluids are produced or prepared in a way that ensures that the amount of carbohydrate-derived toxins are kept as low as possible, see for example WO 93/09820 and WO 97/05852, which hereby are incorporated by reference. Further, the dialysis fluids, food and the nutritional fluids could be treated with substances as described in WO 01/89478 or WO 02/053094, which hereby are incorporated by reference, and further sodium sulphite or N-acetylcysteine. The dialysis fluids, food or nutritional fluids could also be treated, for example, enzymatically to inactivate or inhibit carbohydrate-derived toxins prior to administration. Examples of enzymes are glyoxalase-1 and OPB-9195.

The method of the invention can be practiced with any art-known modes of administration (e.g., dialysis, oral or injections) by which a product containing reduced amount of carbohydrate-derived toxins can be administered to a subject for the purpose of reducing decline of renal function. The administration of any agents which can remove or reduce the amount of carbohydrate-derived toxins present in the body of a subject also falls within the scope of the present invention. Examples of the agents include without limitation sodium sulfite, N-acetylcysteine, fructosamine-3-kinase, glyoxalase-1, and OPB-9195. See Sakai et al., (2001) Adv. Perit. Dialysis 17:66-70; Delpirre et al. (2000) Diabetes 49:1627-1634; Thornally et al. (2003) Biochem. Soc. Transactions 31:1343-1348; Inagi et al. (2002) Kidney Int. 62:679-687; Miyata et al. (2000) JASN 11:1719-1725.

In one embodiment of the invention the method is performed in association with peritoneal dialysis and comprises administering a peritoneal dialysis solution with reduced amount of carbohydrate-derived toxins into the peritoneal cavity of a patient. In these instances, it is desirable that the concentration of glucose degradation products in the peritoneal dialysis solution is below 50 μM, preferably below 25 μM when the glucose concentration in the dialysis solution is 1.5 w/v %, and below 150 μM, preferably below 70 μM when the glucose concentration is 4 w/v %.

The term “carbohydrate-derived toxins” as used herein, refers to any composition that is formed by carbohydrate degradation. These include, but are not limited to, glucose- or glucose polymer-derived toxins such as acetaldehyde, formaldehyde, glyoxal, methylglyoxal, 3-DG, and 3,4-DGE.

Another embodiment of the present invention is a method of limiting the intake of carbohydrate-derived toxins. This can be accomplished either by administering a product with reduced amount of carbohydrate-derived toxins or administering an agent along with a product, which is capable of removing or reducing such toxins. For example, a variety of oral absorber products are known in the art as listed above that can reduce the concentration of the carbohydrate-derived toxins. These agents can be administered orally before or after food intake, or co-administered with diet to allow removal or inactivation of toxic glucose degradation products in the intestine. Alternatively, food or nutritional fluids can be treated enzymatically (e.g. fructosamine-3-kinase, glyoxalase-1, and OPB-9195) to inactivate carbohydrate-derived toxins therein before uptake.

Additionally, the carbohydrate-derived toxins can be specifically removed from blood circulation using the invention through the dialysis with a fluid containing low amounts of carbohydrate-derived toxins. Further, the method of the invention can be used to reduce or eliminate fructose since fructose is the first product in the degradation of carbohydrates into toxins.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

The terms such as “residual renal function”, “renal function”, and “renal failure” are standard terms well known in the art. For example, the renal function is considered to be normal when the value of creatinine clearance is in the range of 97-137 ml/min/1.73 m² (male) and 88-128 ml/min/1.73 m² (female), and the value of urea clearance is in the range of 64-99 ml/min. Residual GFR prediction using the arithmetic means of renal urea and creatinine clearance was recommended by the 1997 National Kidney Foundation-Dialysis Outcomes Quality Initiative (NKF-DOQI), Clinical practice guidelines for peritoneal dialysis adequacy, and is also included in the European best practice guidelines for hemodialysis (Part 1) published in 2002 (Nephrol Dial Transplant 2002, 17 (Suppl 7): 7-15). According to DOQ1 Guideline 6, Residual renal function (RRF), which can provide a significant component of total solute and water removal, should be assessed by measuring the renal component of Kt/Vᵤᵣₑₐ (Kᵣt/Vᵤᵣₑₐ) and estimating the patient's glomerular filtration rate (GFR) by calculating the mean of urea and creatinine clearance.

The term, “reduced” as used in the “reduced amount of carbohydrate-derived toxins”, means that a product useful for the present invention has been treated such that the concentration of the carbohydrate-derived toxins present therein is lower after the treatment than that of the untreated product. For example, the concentration of the carbohydrate-derived toxins present in the peritoneal dialysis fluid useful for the invention is below 50 μM when the glucose concentration in the solution is about 1.5 w/v %, or below 150 μM when the glucose concentration is about 4 w/v %. The amount of the carbohydrate-derived toxins present in a product useful to practice the invention can be reduced by employing various methods disclosed herein, e.g., the products are produced in a manner to contain less toxins, treatment of the product with specific agents capable of removing or reducing such toxins, enzyme treatment, co-administration of an agent capable of removing or reducing such toxins along with a diet or before or after the diet etc.

The term, “glucose-polymer”, refers to a polymer of glucose units and includes oligomers and the like (e.g. icodextrin).

Disclosed herein is a method of reducing decline of renal function of a subject or preserving residual renal function of a patient with impaired renal function by administering a product with reduced amount of carbohydrate-derived toxins or a product capable of removing or reducing such toxins in the body of such a patient.

The present invention is particularly useful for treating a patient suffering from renal failure to preserve the residual renal function. The residual renal function is an important metabolic parameter even though it is below 5% of normal renal function. Prospective clinical studies (10) showed that each 1 ml/min residual renal function can be correlated to objective therapeutic benefit and prolonged stay on PD therapy. As described above, the level of residual renal function is a strong predictor of morbidity and mortality in peritoneal dialysis patients [Nolph et al. (10), Szeto et al. (12), Wang et al. (13), Bargman et al. (7)].

The inventors herein are the first to discover the methods of preserving the residual renal function—even at the level of <5% of normal renal function—by controlling the uptake of carbohydrate degradation products (i.e., glucose degradation products in certain metabolic situations, i.e. renal disease). Based on the findings disclosed below, it may be beneficial in certain subjects to start dialysis interventions even earlier—best at a hypothetical equilibrium situation—when the residual renal capacity is no longer sufficient to compensate against accumulation of carbohydrate-derived toxins. Reducing the decline of renal function or even preserving the residual renal function will accordingly reduce the mortality, i.e. with respect to infection frequency, prolonged survival, reduced risk for cardiovascular complication, prolonged survival before bridging to other dialysis modes, improved quality of life due to non-restriction in food and fluid up-take and improved conditioning for transplantation.

When the method according to the invention to reduce decline of renal function or to preserve residual renal function is practiced with a routine PD treatment, there is no need for an additional medication. Further, with the finding that reduction of the load of carbohydrate-derived toxins has an effect on reduction of the decline in renal function (e.g. uptake in the range or equivalent to a PD therapy), elimination or inactivation along the carbohydrate toxin pathway can be advantageously applied for therapeutic purposes, e.g. by oral scavengers, by reduced uptake from diet, by specifically prepared food, by pro-biotic food, by pre-purified carbohydrate containing liquids and so forth. Such therapeutic modes would belong to therapies for the pre-ESRD phase. It could be specifically advantageous to start PD therapy in lower doses earlier and achieve by this a higher level (e.g. above 6 ml/min) of residual renal function.

The following studies were performed to demonstrate that the administration of a peritoneal dialysis fluid with a lower concentration of GDPs to a group of patients (end stage renal disease patients, treated with PD) indeed reduces the decline of the residual renal function. A total of thirty one patients were divided into two groups, one standard group provided with a standard CAPD-solution and one trial group provided with a CAPD-solution containing low GDPs. The study was done in prospective randomisation to exclude any bias from different perspectives, i.e. doctor, patient, or nursing personnel.

The standard 1.5 w/v % glucose PD-solution contained >350 μM GDPs and the low GDP containing PD-solution contained <25 μM GDPs. The low GDP containing PD-solution was the PD-solution sold under the trademark Gambrosol™ trio, by the company Gambro Renal Products.

The patients visited the study centre at 4-6 week intervals and the residual renal function was recorded at each visit.

The patients collected and brought the 24-hour urine to the medical visit. The urine volume and the concentration of urea and creatinine in urine and plasma were determined according to the methods well known in the art. The residual renal function was calculated with the formula given below. At time intervals T1, T6, T12 and T18, at which time the peritoneal transport characteristics were determined using the PDC™ computer program, sold by Gambro Renal Products, the residual renal function (RRF) was automatically calculated by the program (formula also given below). For simplification purposes, the value for the corresponding RRF can be taken from the PDC program.

RRF Calculation Formula

$$RRF = \frac{Clearance ( urea ) + Clearance ( creatinine )}{2}$$

wherein the clearance of urea or creatinine (in ml/min normalized to 1.73 m² BSA) was calculated as follows:

$$K = \frac{C ( u ) \times V ( u ) \times 1.73}{C ( p ) \times 24 \times 60 \times BSA}$$

wherein

BSA Body surface area; calculated value taken from PDC at Time T1

K Clearance for urea or creatinine

C Concentrations in urine (u) or plasma (p)

V Volume (residual 24h-urine)

The results shown in this application were gathered from a standard group with 131 single data from 13 patients and a trial group with 230 single data from 23 patients.

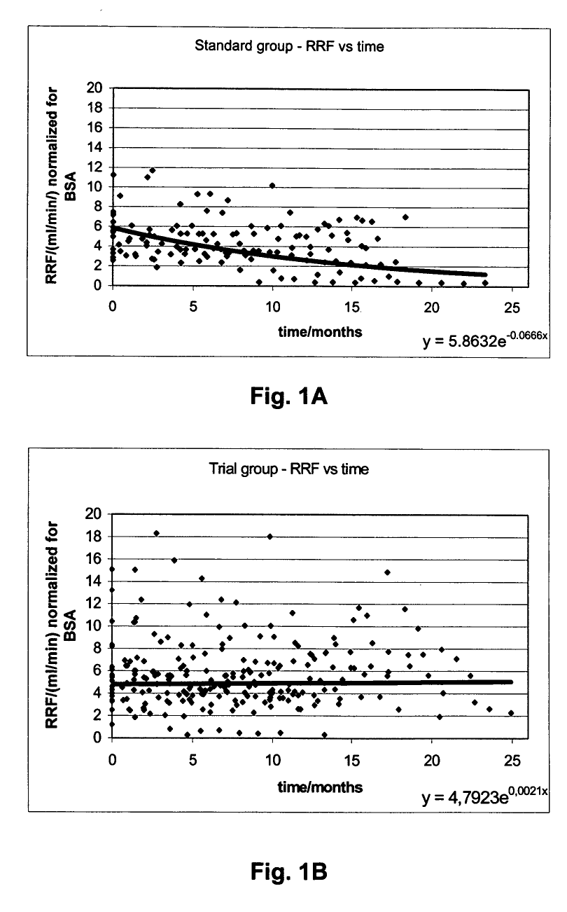

The results are shown in FIGS. 1A and 1B. As evident in the Figures, there is a significant difference between the standard group and the trial group, i.e., the plot for the standard group shows a significant negative slope with declining residual renal function over time, while the trial group shows no significant change in the residual renal function. Statistical data analysis was performed using the data processing system RS/1 (V. 6.0.1; BBN Software Products Co., Cambridge, Mass., USA). Analysis of covariance and linear data modelling, assuming separate slopes (change in residual renal function over time) for the two different PD fluids and applying unweighted robust calculations, revealed the following:

(1) After adjusting for the predictor variable time, there is strong evidence that the type of PD fluid affects residual renal function with an error probability of 0.014%.

(2) The change in residual renal function for each unit increase in time (slope) is −0.1434 (ml/min)/month (95% confidence interval −0.2166 to −0.0702 (ml/min)/month) for standard PD fluid (high-GDP) and 0.0366 (ml/min)/month (95% confidence interval −0.0192 to 0.0925 (ml/min)/month) for Gambrosol™ trio (low-GDP).

(3) The slopes given in (2) are significantly different (p=0.0001).

(4) The slope given in (2) for the low-GDP fluid is not significantly different from zero (p=0.198), whereas the slope for the high-GDP fluid is significantly different from zero (p=0.0001).

(5) The difference between the levels of residual renal function in the two study groups becomes significant (p=0.0118) after 6 months. At 12 months the significance level is at p=0.0001.

In an alternative statistical approach a linear mixed model was successfully fitted to the residual renal function (RRF) data. SAS software was used for this analysis. It includes the lineal component, and a compound symmetry matrix for the covariance of measurements from the same patients.

The whole data set provided enough power to the model to detect a highly significant difference in the slope of the decline of RRF between treatment with high-GDP PD fluid vs. low-GDP PD fluid (0.107±0.016 (ml/min)/month vs. 0.035±0.030 (ml/min)/month, p<0.0001). The decline of RRF over time in the study group using low-GDP fluid is significantly closer to zero than in the group using high-GDP fluid (p=0.0161).

The studies described above indicate that the effects observed are due to the reduced amount of GDPs in the peritoneal dialysis fluid as this is the only difference between the peritoneal dialysis fluids given to the standard group and the trial group.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the invention is to be limited only to the claims except as precluded by the prior art. Moreover, the invention as disclosed herein, may be suitably practiced in the absence of the specific elements which are disclosed herein.

Materials and Methods

The following examples are provided to illustrate how various glucose derived toxins can be measured.

| | | | Molecular |
|---|---|---|---|
| | Abbrev. | Formula | weight |
| Acetaldehyde | | C₂H₄O | 44.1 |
| Formaldehyde | | CH₂O | 30.0 |
| Glyoxal | | C₂H₂O₂ | 58.0 |
| Methylglyoxal | | C₃H₄O₂ | 72.1 |
| 3-Deoxyglucosone | 3-DG | C₆H₁₀O₅ | 162.1 |
| 3,4-Dideoxyglucosone | 3,4-DGE | C₆H₈O₄ | 144.1 |
| 5-hydroxymethylfuraldehyde | 5-HMF | C₆H₆O₃ | 126.1 |

Chemicals B:

Acetaldehyde; Fluka (Germany) catalogue nr. 0007, lot nr. 417869/1 31901.

Formaldehyde (37%); Sigma Chemical catalogue nr. F1635, lot nr. 11K1269.

Glyoxal (30%); Merck (Germany) catalogue nr. 820610, lot nr. 9124664.

Methylglyoxal (40%); Sigma Chemical catalogue nr. M-0252, lot nr. 100H0429.

3-Deoxyglucosone; TRC (Toronto, Ohio) catalogue nr. D239150, lot nr 11-WG-160-1.

3,4-Dideoxyglucosone; Synthesized on request by TRC (Toronto, Ohio).

5-HMF (99%9 Sigma Chemical (USA) catalogue nr. H-9877 lot nr. 127H3428

Acetonitril; Merck (Germany) catalogue nr. 1.00030, lot nr. 1029930216

Methanol; Merck (Germany) catalogue nr. 1.06018, lot nr. K30155218203

Natrium phosphate; Merck (Germany) catalogue nr. 1-7157, lot nr. A191846946

2,3-diaminonaphtalene; Sigma Chemicals (USA) catalogue nr. D2757, lot nr. 101K1087

1,2-phenylenediamine; Sigma Chemicals (USA) catalogue nr. P9029, lot nr. 11K5312

2,4-dinitrophenylhydrazine; Sigma Chemicals (USA) catalogue nr. D2630, lot nr. 2506E

Equipment: One HPLC (ID. RR025+DAD RR0526) system consisted of an liquid chromatograph series 1100 from Agilent Technologies equipped with an autosampler and an UV-detector. Agilent Technologies Chem Station software rev.A.08.03, NT 4.0 was used for the data handling.

Determination of 3-DG (SF/AKE 199): 3-DG was determined as quinoxaline using 2,3-diaminonaphtalene as derivative reagent. The samples were diluted 50 times prior to analysis. The standards were prepared in the range 1-6 μM. Standards and samples were prepared by adding 100 μl 0,1% 2,3-diaminonaphtalene to 1 ml sample and incubated for 16 hours in room temperature and dark. The analytical column was a Water Symmetry C18 column (5 μm, 25 cm×4.6 mm). The elution of the substance was performed at constant flow rate of 1.0 ml/min by using a gradient of acetonitrile/water. The concentration (%) was initially 25/75 and 12 minutes later 25/75, at 15 minutes 60/40 and at the gradient stop 30 minutes 60/40. The wavelength was set at 268 nm and the injected volume was 20 μl. The limit of quantitation was 1 μM.

Determination of 5-HMF (SF/AKE 079 rev.2): The samples for the determination of 5-HMF was diluted 5 times prior to analysis. The 5-HMF standards were prepared in the range 0.8-15.9 μM. The analytical column was a Supelco C18 column (5 μm, 15 cm×4.6 mm). The elution of the substances was performed at constant flow of 1.2 ml/min using 10% acetonitrile and 90% 0.05 M sodiumphosphate. The wavelenght was set at 283 nm and the injected volume was 100 μl. The limit of quantitation for 5-HMF was 0.8 μM.

Determination of acetaldehyde and formaldehyde (SF/AKE 065 rev.4): The samples for the determination of acetaldehyde was diluted 20 times prior to analysis. Acetaldehyde and formaldehyde were prepared as hydrazone derivatives using 2,4-DNPH as derivative reagent. The standards were prepared in the range 1.1-11.4 μM acetaldehyde, and 1.7-16.7 μM formaldehyde. Standards and samples were prepared by adding 2 ml 0.08% 2,4-DNPH to 4 ml of each sample. The samples were concentrated on a solid phase extraction C18 column (Bond Elut LRC 200 mg/3 ml) and after rinsing with water, eluted with 1.6 ml acetonitrile. The analytical column was a Supelco C18 column (5 μm, 15 cm×4,6 mm). The elution of the substances was performed at constant flow of 1,7 ml/min by using a linear gradient of acetonitrile/water. The concentration (%) was initially 35/65 and at the gradient stop 12 minutes later 80/20. The wavelenght was set at 240 nm and the injected volume was 20 μl. The limit of quantitation was for acetaldehyde 1.1 μM and for formaldehyde 1.7 μM.

Determination of 3,4-dideoxyglucosone(3,4-DGE) (SF/MBR 004): The samples for the determination of 3,4-DGE was diluted 20 times prior to analysis. The 3,4-DGE standards were prepared in the range 0.3-3.5 μM. The analytical column was a Genesis LC 18, (4 μm, 25 cm×4.6 mm, Sorbent). The elution of the substance was performed at constant flow of 1.0 ml/min by using a gradient of methanol/water. The concentration (%) was initially 10/90 and 6 minutes later 10/90, at 8 minutes 100% methanol, at 16 minutes 100% methanol and at the gradient stop 25 minutes 10/90. The wavelength was set at 230 nm and the injected volume was 100 μl. The limit of quantitation for 3,4-DGE was 0.3 μM.

Determination of glyoxal and methylglyoxal (SF/AKE 198): Glyoxal and methylglyoxal were determined as quinoxalines using 1,2-phenylenediamine. The standards were prepared in the range 3.5-51.7 μM glyoxal and 2.8-41.7 μM methylglyoxal. Standards and samples were prepared by adding 0.6 ml 0.4% 1,2-phenylenediamine to 1 ml of each sample. The analytical column was a Supelco C18 column (5 μm, 25 cm×4.6 mm). The elution of the substances was performed at constant flow of 1.0 ml/min using a mobile phase of initial 25% acetonitrile and 75% 0.05 M sodiumphophate. At the gradient at 6 minutes the mobile phase was 30/70 and at the gradient stop 9 minutes 25/75. The wavelength was set at 312 nm and the injected volume was 20 μl. The limit of quantitation was for glyoxal 3.5 μM and for methylglyoxal 2.8 μM.

### REFERENCES

1. Lindén, T. P., et al.: 3,4-Dideoxyglucosone-3-ene (3,4-DGE), The Cytotoxic Glucose Degradation Product in PD Fluids? J. Am. Soc. Nephrol. 12:432A, 2001.

2. Kato, F., et al.: Immunosuppressive Effects of 3,4-dideoxyglucosone-3-ene, An Intermediate in the Maillard Reaction. J. Agri. Food Chem. 42:2068-2073, 1994.

3. Lindén, T. P., et al.: 3,4-dideoxyglucosone-3-ene (3,4-DGE), A Cytotoxic Glucose Degradation Product in Fluids for Peritoneal Dialysis. Kidney Int. 62: 697-703, 2002.

4. Nakayama, M., et al.: Immunohistochemical Detection of Advanced Glycosylation End-products in the Peritoneum and its Possible Pathophysiological Role in CAPD. Kidney Int. 51(1):182-186, 1997.

5. Zeier, M., et al.: Glucose Degradation Products in PD fluids: Do They Disappear From the Peritoneal Cavity and Enter the Systematic Circulation? Kidney Int. 63:298-305, 2003.

6. Lysaght, M. J., et al.: The Influence of Dialysis Treatment Modality on the Decline of Remaining Renal Function. ASAIO Transactions 37:598-604,1991.

7. Bargman, J. M., et al.: Relative Contribution of Residual Renal Function and Peritoneal Clearance to Adequacy of Dialysis: a Reanalysis of the CANUSA Study. J. Am. Soc. Nephrol. 12: 2158-2162, 2001.

8. Menon, M. K., et al.: Long-term Blood Pressure Control in a Cohort of Peritoneal Dialysis Patients and Its Association with Residual Renal Function. Nephrol. Dial. Transplant 16(11):2207-2213, 2001.

9. Shemin, D., et al.: Residual Renal Function in a Large Cohort of Peritoneal Dialysis Patients: Change Over Time, Impact on Mortality and Nutrition. Perit. Dial. Int. 20(4):439-444, 2000.

10. Venkataraman, V., et al.: Preservation of Residual Renal Function—an Important Goal. Perit. Dial. Int. 20: 392-395, 2000.

11. Rocco, M. V., et al.: Risk Factors for Early Mortality in US Peritoneal Dialysis Patients: Impact Residual Renal Function. Perit. Dial. Int. 22:371-379, 2002.

12. Szeto, C., et al.: Independent Effects of Residual Renal Function and Dialysis Adequacy on Nutritional Status and Patient Outcome in Continuous Ambulatory Peritoneal Dialysis. Am. J. Kidney Dis. 34(6):1056-1064, 1999.

13. Wang, A. Y., et al: A Novel Association Between Residual Renal Function and Left Ventricular Hypertrophy in Peritoneal Dialysis Patients. Kidney Int'l. 62:639-647, 2002.

All references cited in the present application are incorporated in their entirety herein by reference to the extent not inconsistent herewith.

1. A method of reducing decline of renal function of a subject or preserving residual renal function of a subject with impaired renal function, wherein said method comprises administering a product with reduced amount of carbohydrate-derived toxins into the body of the subject.

2. A method according to claim 1, wherein the carbohydrate-derived toxins are glucose- or glucose polymer-derived toxins.

3. A method according to claim 2, wherein the glucose- or glucose polymer-derived toxins are glucose degradation products.

4. A method according to claim 1, wherein said product is a dialysis solution with reduced amount of carbohydrate-derived toxins.

5. A method according to claim 4, wherein said product is a peritoneal dialysis solution with reduced amount of carbohydrate-derived toxins administered into the peritoneal cavity of a subject.

6. A method according to claim 5, wherein the carbohydrate-derived toxins are glucose- or glucose polymer-derived toxins.

7. A method according to claim 6, wherein the glucose- or glucose polymer-derived toxins are glucose degradation products.

8. A method according to claim 7, wherein the concentration of the glucose degradation products in the peritoneal dialysis solution is below 50 μM, when the glucose concentration in the solution is about 1.5 w/v %.

9. A method according to claim 8, wherein the concentration of the glucose degradation products in the peritoneal dialysis solution is below 25 μM, when the glucose concentration in the solution is about 1.5 w/v %.

10. A method according to claim 7, wherein the concentration of the glucose degradation products in the peritoneal dialysis solution is below 150 μM, when the glucose concentration in the solution is about 4 w/v %.

11. A method according to claim 10, wherein the concentration of the glucose degradation products in the peritoneal dialysis solution is below 70 μM, when the glucose concentration in the solution is about 4 w/v %.

12. A method according to claim 1, wherein said product is a foodstuff or nutritional fluid with reduced amount of carbohydrate-derived toxins.

13. A method according to claim 12, wherein said food or nutritional fluid further comprises an agent capable of removing or inactivating the carbohydrate-derived toxins in the intestine.

14. A method according to claim 13, wherein the agent is capable of absorbing the carbohydrate-derived toxins in the intestine.

15. A method according to claim 12, wherein said food or nutritional fluid contains no or reduced amount of fructose.

16. A method according claim 12, wherein said food or nutritional fluid is enzymatically treated to inactivate carbohydrate-derived toxins therein.

17. A method of treating a patient suffering from renal failure, wherein said method comprises administering a peritoneal dialysis solution with reduced amount of carbohydrate-derived toxins into the peritoneal cavity of the patient such that the decline of renal function is reduced or the residual renal function is preserved.

18. A method according to claim 17, wherein said carbohydrate-derived toxins are glucose- or glucose polymer-derived toxins.

19. A method according to claim 18, wherein said glucose- or glucose polymer-derived toxins are glucose degradation products.

20. A method according to claim 19, wherein the concentration of glucose degradation products in the peritoneal dialysis solution is below 50 μM, when the glucose concentration in the solution is about 1.5 w/v %.

21. A method according to claim 20, wherein the concentration of glucose degradation products in the peritoneal dialysis solution is below 25 μM, when the glucose concentration in the solution is about 1.5 w/v %.

22. A method according to claim 19, wherein the concentration of glucose degradation products in the peritoneal dialysis solution is below 150 μM, when the glucose concentration in the solution is about 4 w/v %.

23. A method according to claim 22, wherein the concentration of glucose degradation products in the peritoneal dialysis solution is below 70 μM, when the glucose concentration in the solution is about 4 w/v %.

24. A method of performing peritoneal dialysis wherein said method comprises administering a peritoneal dialysis solution with reduced amount of carbohydrate-derived toxins into the peritoneal cavity of a subject such that the decline of renal function is reduced or residual renal function is preserved.

25. A method according to claim 24, wherein said carbohydrate-derived toxins are glucose- or glucose polymer-derived toxins.

26. A method according to claim 25, wherein said glucose- or glucose polymer-derived toxins are glucose degradation products.

27. A method according to claim 26, wherein the concentration of glucose degradation products in the peritoneal dialysis solution is below 50 μM, when the glucose concentration in the solution is about 1.5 w/v %.

28. A method according to claim 27, wherein the concentration of glucose degradation products in the peritoneal dialysis solution is below 25 μM, when the glucose concentration in the solution is about 1.5 w/v %.

29. A method according to claim 26, wherein the concentration of glucose degradation products in the peritoneal dialysis solution is below 150 μM, when the glucose concentration in the solution is about 4 w/v %.

30. A method according to claim 29, wherein the concentration of glucose degradation products in the peritoneal dialysis solution is below 70 μM, when the glucose concentration in the solution is about 4 w/v %.

31. A method of reducing decline of renal function of a subject or preserving residual renal function of a subject with impaired renal function, wherein said method comprises administering a product capable of removing or reducing carbohydrate-derived toxins present in the body of the subject such that the decline of renal function is reduced or the residual renal function is preserved.

32. The method of claim 31 wherein the product is an agent capable of absorbing the carbohydrate-derived toxins.

33. The method of claim 31 wherein the product is an enzyme capable of degrading the carbohydrate-derived toxins.
